(19) United States
(12) Patent Application Publication
(10) Pub. No.: US 2021/0052823 A1
(43) Pub. Date: Feb. 25, 2021

(54) RECEPTACLE FOR SEVERING AND RETAINING THE NEEDLE TIP OF A HYPODERMIC SYRINGE

(71) Applicant: Steven F. McNelley, Wrentham, MA (US)

(72) Inventor: Steven F. McNelley, Wrentham, MA (US)

(21) Appl. No.: 16/989,195

(22) Filed: Aug. 10, 2020

Related U.S. Application Data
(60) Provisional application No. 62/889,202, filed on Aug. 20, 2019.

(51) Int. Cl. A61M 5/32; A61B 50/30

(52) U.S. Cl. CPC A61M 5/321; A61B 50/3001

(57) ABSTRACT

A receptacle for severing and retaining the needle tip of a hypodermic syringe includes a highly compact outer housing, which is shaped to define a substantially enclosed interior cavity, and a knife blade fixedly mounted within the interior cavity. The outer housing includes a flattened base and a hollow, hemicylindrical cover that are permanently secured together. In operation, the needle tip of a used syringe is inserted through coaligned, tear-shaped openings formed in both the cover and the knife blade. Through manipulation of the hypodermic syringe, the needle tip is drawn into contact with a sharpened, beveled edge that immediately defines the opening in the knife blade. Through the application of sufficient force against the sharpened edge, the needle tip is severed from the remainder of the syringe and thereby remains safely and securely retained within the interior cavity.

### CROSS-REFERENCE TO RELATED APPLICATIONS

The present application claims the benefit under 35 U.S.C. 119(e) of U.S. Provisional Patent Application No. 62/889,202, which was filed on Aug. 20, 2019 in the name of Steven F. McNelley, the disclosure of which is incorporated herein by reference.

### FIELD OF THE INVENTION

The present invention relates generally to hypodermic syringes and, more particularly, to containers designed for the safe disposal of used hypodermic syringes.

### BACKGROUND OF THE INVENTION

A hypodermic syringe is a device commonly utilized in the healthcare industry to, inter alia, inject substances into the human body (e.g., insulin or other forms of medicine) or extract fluids from the human body (e.g., blood samples). To prevent the transmission of various types of bloodborne pathogens amongst multiple people, hypodermic syringes are designed strictly for utilization as single-use devices.

A hypodermic syringe typically comprises a hollow plastic tube, or barrel, from which extends a hollow metal needle, or cannula, in fluid communication therewith. An externally-accessible plunger, or piston, is adapted for coaxial displacement within the internal cavity of the hollow barrel. In this manner, insertion of the plunger into the barrel can be used to deliver medicine retained therein into the metal needle and out through its open tip. Similarly, withdrawal of the plunger from the barrel creates a vacuum which draws fluid from the open tip of the metal needle and into the interior of the barrel.

To facilitate insertion of the needle into a patient, the tip of the needle is typically beveled to form a sharped point. To prevent inadvertent puncture by the sharpened point, a hypodermic needle is typically provided with a removable cap to temporarily cover the sharpened point.

For a multitude of reasons, it is critical that used hypodermic syringes be disposed of in a safe and reliable manner. Most notably, because the syringe cap is designed to be readily removed from the needle tip, a used syringe is capable of being either intentionally or unintentionally reused by multiple people, thereby creating a significant biomedical hazard.

Hypodermic syringes are often utilized by medical professionals in environments which are well-suited to dispose of used syringes in a safe and reliable fashion. Notably, medical professionals typically dispose of used syringes into a sharps container which, in turn, is periodically emptied by professional medical waste disposal services.

However, patients are increasingly required to self-administer medication (e.g., insulin) using hypodermic syringes at unforeseen times and in unfamiliar environments. Accordingly, it is often difficult for patients to discard used needles in a safe and secure fashion.

In fact, it has been found that patients often dispose of used hypodermic syringes directly into trash receptacles or other insecure containers when designated syringe disposal containers are not immediately available. As a result, the improper disposal of used hypodermic syringes by the general public creates a substantial biohazard, as the contaminated needle tip of a used syringe is susceptible to penetration into the skin of different people.

### SUMMARY OF THE INVENTION

It is an object of the present invention to provide a new and improved receptacle for collecting used hypodermic syringes.

It is another object of the present invention to provide a receptacle as described above which collects used hypodermic syringes in a safe and secure fashion.

It is yet another object of the present invention to provide a receptacle as described above which is highly compact and portable.

It is yet still another object of the present invention to provide a receptacle as described above which is simple in construction, easy to operate, and inexpensive to implement.

Accordingly, as one feature of the present invention, there is provided a receptacle for severing and retaining the needle tip of a hypodermic syringe, the receptacle comprising (a) an outer housing shaped to define an interior cavity, and (b) a knife blade mounted within the interior cavity of the outer housing, (c) wherein the knife blade is adapted to fragment the hypodermic syringe into a severed needle tip and a tip-less syringe, the severed needle tip being retained securely within the interior cavity of the outer housing.

As another feature of the present invention, there is provided a method for severing and retaining the needle tip of a hypodermic syringe, the method comprising the steps of (a) providing a receptacle comprising an outer housing shaped to define an interior cavity and a knife blade mounted within the interior cavity of the outer housing, each of the outer housing and the knife blade being shaped to define an opening in communication with the interior cavity, the opening in the knife blade being immediately defined by a sharpened edge, (b) inserting the needle tip of the hypodermic syringe through the opening in the outer housing, through the opening in the knife blade, and into the interior cavity, and (c) manipulating the hypodermic syringe into contact with the sharpened edge in the knife blade so as to fragment the hypodermic syringe into a severed needle tip and a tip-less syringe, with the severed needle tip remaining inside the interior cavity in the outer housing.

Various other features and advantages will appear from the description to follow. In the description, reference is made to the accompanying drawings which form a part thereof, and in which is shown by way of illustration, an embodiment for practicing the invention. The embodiment will be described in sufficient detail to enable those skilled in the art to practice the invention, and it is to be understood that other embodiments may be utilized and that structural changes may be made without departing from the scope of the invention. The following detailed description is therefore, not to be taken in a limiting sense, and the scope of the present invention is best defined by the appended claims.

### BRIEF DESCRIPTION OF THE DRAWINGS

In the drawings, wherein like reference numerals represent like parts;

FIG. 1 is a top perspective view of a first embodiment of a receptacle for severing and retaining the needle tip of a used hypodermic syringe, the receptacle being constructed according to the teachings of the present invention;

FIGS. 2(a) and 2(b) are exploded, top perspective and exploded, bottom perspective views, respectively, of the receptacle shown in FIG. 1;

FIG. 3 is an enlarged, front perspective view of the knife blade shown in FIG. 2(a);

FIG. 4 is a section view of the receptacle shown in FIG. 1, taken along lines 4-4;

FIG. 5 is a top perspective view of a second embodiment of a receptacle for severing and retaining the needle tip of a used hypodermic syringe, the receptacle being constructed according to the teachings of the present invention;

FIGS. 6(a) and 6(b) are exploded, top perspective and exploded, bottom perspective views, respectively, of the receptacle shown in FIG. 5;

FIG. 7 is a section view of the receptacle shown in FIG. 5, taken along lines 7-7;

FIG. 8 is an enlarged, fragmentary view of the receptacle shown in FIG. 7, which is useful in understanding the snap-fit relationship between the cover and the base;

FIG. 9 is a fragmentary bottom perspective view of the receptacle shown in FIG. 5; and

FIGS. 10(a) and 10(b) are top perspective and bottom perspective views, respectively, of a modification to the cover shown in FIG. 5.

### DETAILED DESCRIPTION OF THE INVENTION

### Needle Tip Receptacle 11

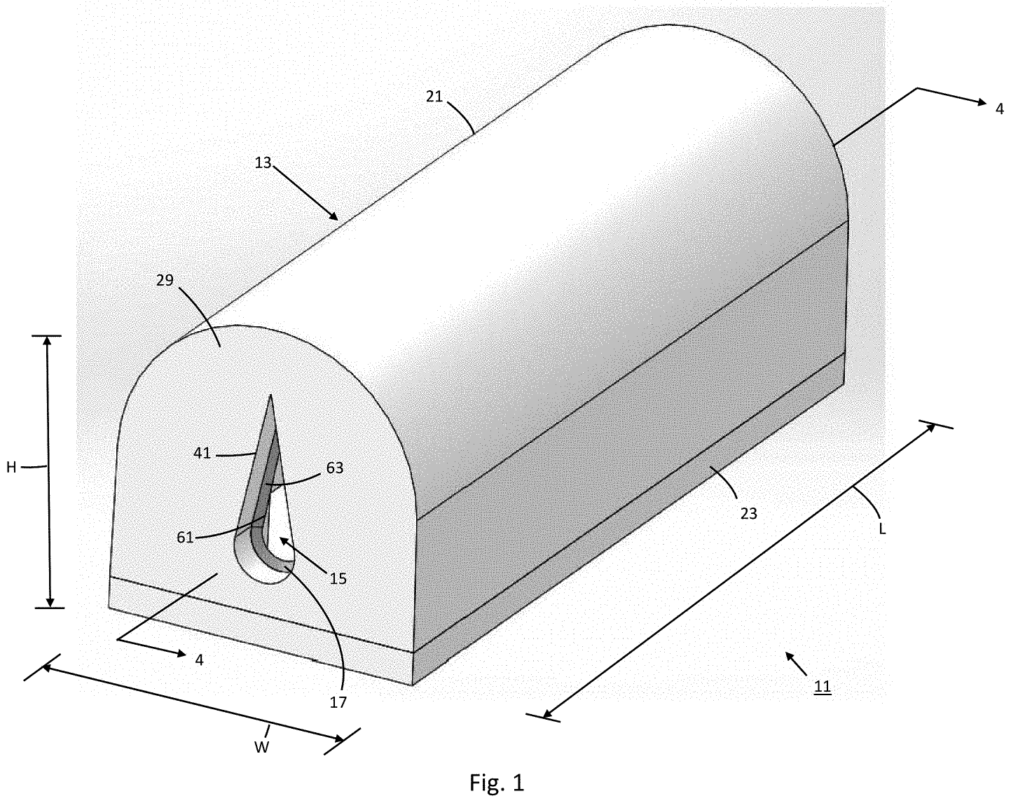

Referring now to FIG. 1, there is shown a receptacle for severing and retaining the needle tip of at least one hypodermic syringe, the receptacle being constructed according to the teachings of the present invention and identified generally by reference numeral 11. As will be explained in detail below, receptacle 11 is uniquely designed to provide a highly portable, safe and secure means for preventing the subcutaneous puncture of a person with a used hypodermic syringe.

In the description that follows, receptacle 11 is described primarily in connection with the severing and retaining of the needle tip from a hypodermic syringe. However, it should be noted that receptacle 11 is not limited to use with hypodermic syringes. Rather, it is to be understood that receptacle 11 could be modified, as needed, for use in severing and retaining the sharpened tips of other single-use, needle-based, medical tools which are principally designed to penetrate the skin, such as blood lancets used in glucose monitoring applications.

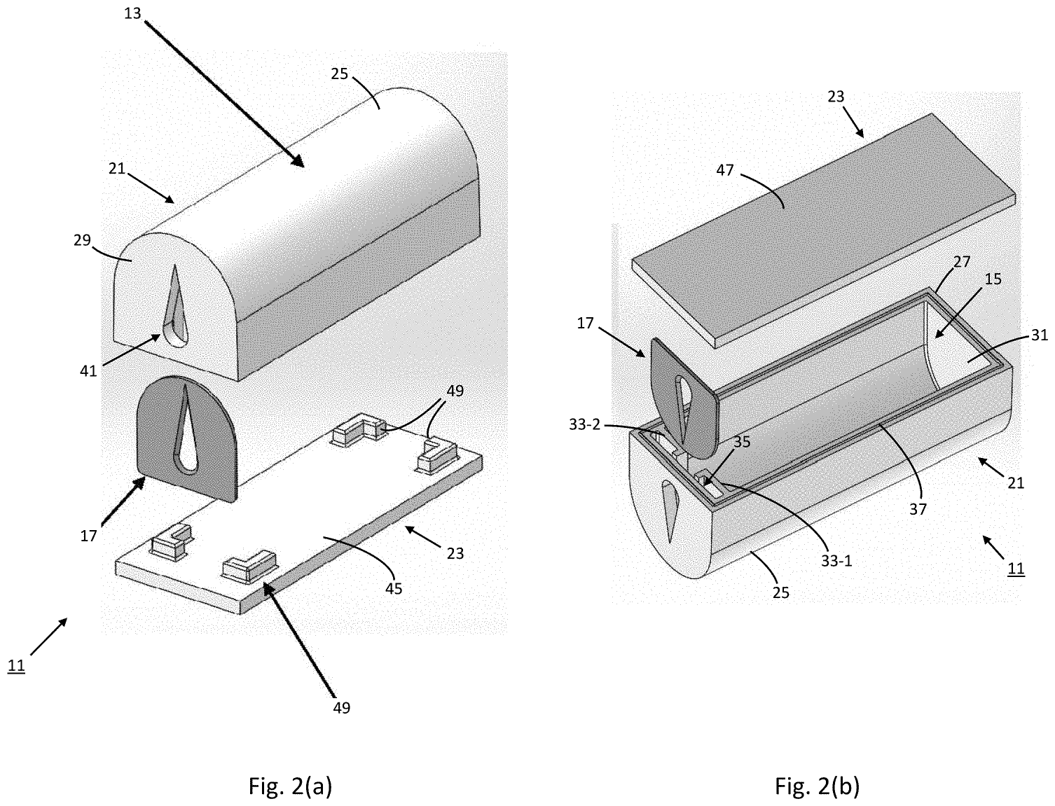

As seen in FIGS. 1, 2(a) and 2(b), needle tip receptacle 11 comprises (i) an outer housing 13, which is shaped to define a substantially enclosed interior cavity 15, and (ii) a knife blade 17 disposed within interior cavity 15. As will be explained further in detail below, housing 13 is adapted to receive the needle tip of a used hypodermic syringe. Through manipulation of the syringe, the needle can be brought into direct contact against blade 17. In this manner, the needle tip can be effectively severed from the remainder of the syringe and, in turn, securely retained within housing 13 so as to prevent any further skin puncture, which is the principal object of the present invention.

Housing 13 is a compact, generally stick-shaped member which is constructed of a rigid and durable material, such as plastic. Preferably, housing 13 has an overall length L of approximately 1.625 inches, an overall width W of 0.625 inches, and an overall height H of approximately of 0.568 inches, the pocket-sized nature of housing 13 rendering receptacle 11 highly portable and therefore readily available for use when needed.

Housing 13 is represented herein as having a two-piece construction comprising a hollow, hemicylindrical cover 21 and a flat, plate-like base 23 which together define interior cavity 15 upon assembly. As will be explained further below, cover 21 is preferably permanently secured to base 23 upon assembly to ensure that any used needle tips retained within interior cavity 15 are completely inaccessible.

Hemicylindrical cover 21 is a generally hollow member which comprises a rounded top surface 25, an open bottom surface 27, a front face 29, and a rear face 31. As seen most clearly in FIG. 2(b), a pair of L-shaped projections 33-1 and 33-2 protrude from opposing interior sides of top surface 25 proximate front face 29 so as to define a narrow slot 35 therebetween. As can be appreciated, slot 35 is appropriately dimensioned to fittingly receive knife blade 17 upon assembly, as will be explained further in detail below.

A continuous ultrasonic weld tab 37, generally V-shaped in transverse cross-section, protrudes from open bottom surface 27. As will be explained further in detail below, weld tab 37 is provided to facilitate the permanent securement of cover 21 onto base 23.

As seen most clearly in FIG. 2(a), a tear-shaped opening 41 is formed in front face 29 of hemicylindrical cover 21. As can be appreciated, opening 41 is appropriately dimensioned to receive the needle of a used hypodermic syringe. In this manner, the needle can be manipulated into contact with knife blade 15 and severed, such that the used needle tip remains securely stored within interior cavity 15.

Base 23 is a unitary, plate-like member which includes a generally flat top surface 45 and a generally flat bottom surface 47. An L-shaped alignment boss, or post, 49 protrudes upward from each corner of top surface 45. Bosses 49 are configured to align directly inside top surface 25 of cover 21 and therefore serve to facilitate and maintain proper alignment of cover 21 onto base 23 during the process of assembling receptacle 11.

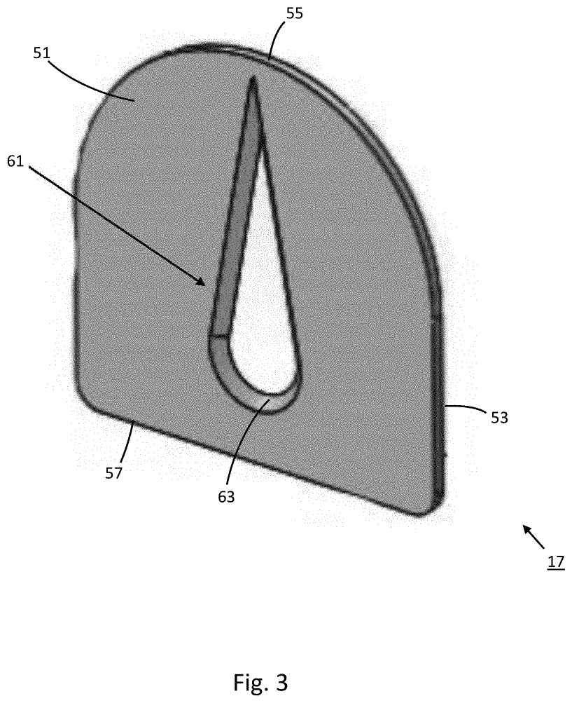

Referring now to FIG. 3, knife blade 17 is preferably constructed as a thin plate of rigid and durable material, such as stainless steel. In the present embodiment, knife blade 17 is shown as a flat, semi-elliptical, metal blank, approximately 0.015 inches in thickness, which includes a flat front surface 51, a flat rear surface 53, a rounded top edge 55, and a flat bottom edge 57.

Although not shown herein, it is to be understood that knife blade 17 may be housed within an outer casing. In this manner, the overall thickness of such a knife blade assembly would be increased to the extent necessary to allow for adequate fixed securement of knife blade 17 within housing 13 upon assembly of receptacle 11.

A tear-shaped opening 61 is formed in knife blade 17, opening 61 having the same general size and shape as opening 41 in cover 21. Accordingly, openings 41 and 61 are in dimensional uniformity when aligned upon assembly of receptacle 11. The peripheral edge 63 which immediately defines opening 61 is preferably razored down to approximately 0.007 inches and is beveled. In this manner, edge 63 is suitably sharpened to allow for the cutting of a metal syringe needle when disposed thereagainst.

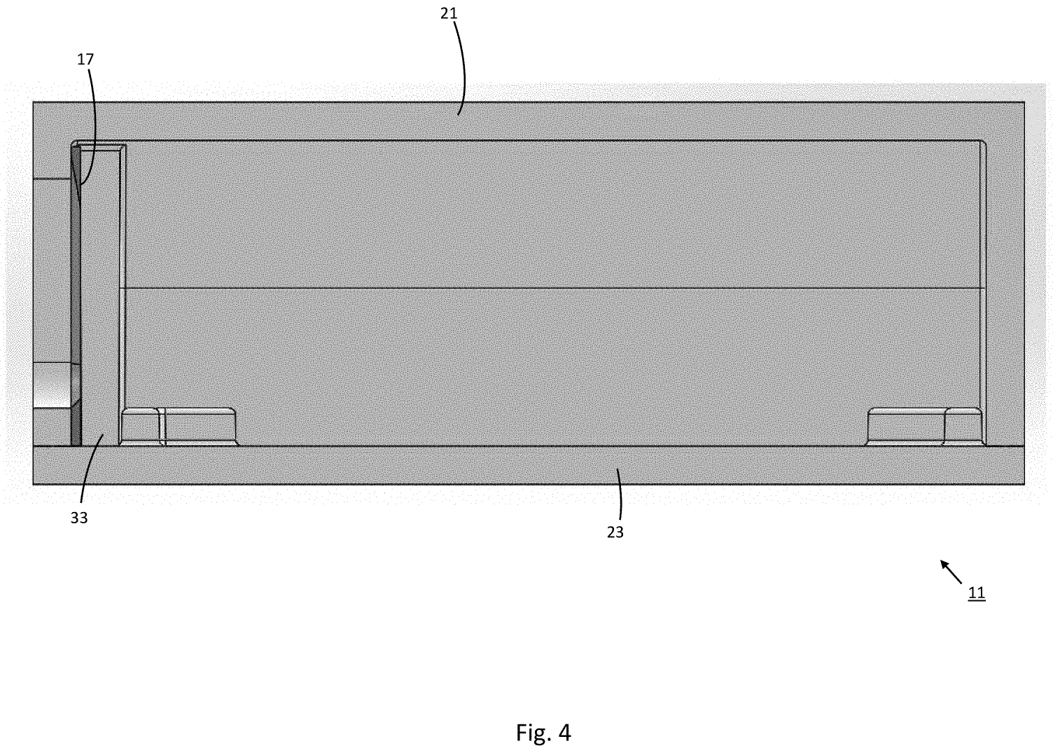

As referenced briefly above, knife blade 17 is dimensioned for fitted insertion into slot 35 in cover 21, with top edge 55 positioned in contact against the interior of rounded top surface 25. Positioned as such, the distal ends of projections 33 abut against rear surface 53 of knife blade 17 and the interior of front face 29 abuts against front surface 51 of knife blade 17. In this manner, knife blade 17 is securely retained within slot 35 in cover 21, as shown in FIG. 4.

With knife blade 17 positioned in slot 35, cover 21 is mounted onto base 23, with L-shaped bosses 49 on base 23 ensuring proper orientation. As referenced earlier, it is envisioned that cover 21 be permanently secured to base 23 as part of the assembly process to ensure that severed needle tips remain safely stored within interior cavity 15.

In the present embodiment shown in FIGS. 1, 2(a) and 2(b), ultrasonic welding is preferably utilized to secure cover 21 to base 23. Notably, weld tab 37 is directly welded to base 23 to permanently secure receptacle 11 in its assembled state. Accordingly, it is envisioned that when receptacle 11 retains a maximum number of severed needle tips (e.g., 10 needle tips), receptacle 11 is set aside for safe disposal and a new receptacle 11 is utilized thereafter.

It should be noted that housing 13 is not limited to ultrasonic welding as means for securing cover 21 to base 23. Rather, it is to be understood that alternative means for securing cover 21 to base 23 could be utilized without departing from the spirit of the present invention.

As an example, an adhesive could be utilized to permanently bond together cover 21 and base 23. Such a bonding agent could be applied directly within the region of contact between cover 21 and base 23 or, in the alternative, applied within specifically designed glue holes (not shown) that are formed in cover 21 and/or base 23.

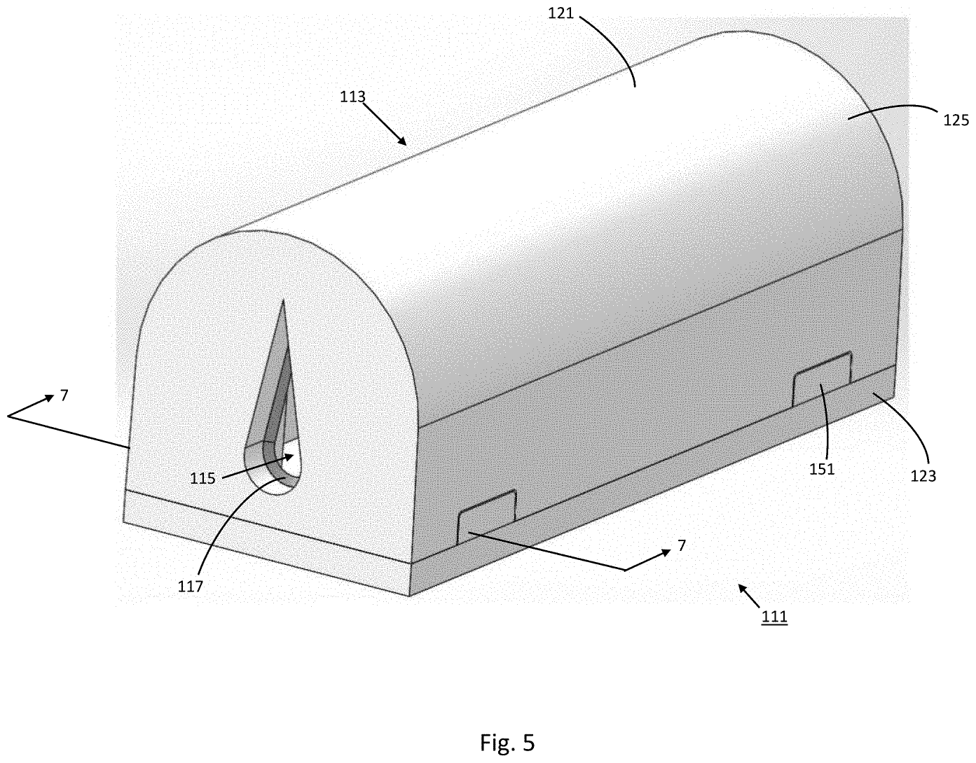

As another example, in FIG. 5, there is shown a second version of needle tip receptacle constructed according to the teachings of the present invention and identified generally by reference numeral 111. Receptacle 111 is similar to receptacle 11 in its general design and function in that receptacle 111 includes (i) an outer housing 113, which is shaped to define a substantially enclosed interior cavity 115, and (ii) a knife blade 117 disposed within interior cavity 115.

Receptacle 111 differs from receptacle 11 in the particular means for securing outer housing 113 in its assembled state. Specifically, as will be described below, receptacle 111 relies principally on a snap fit connection to secure outer housing 113 in its assembled state.

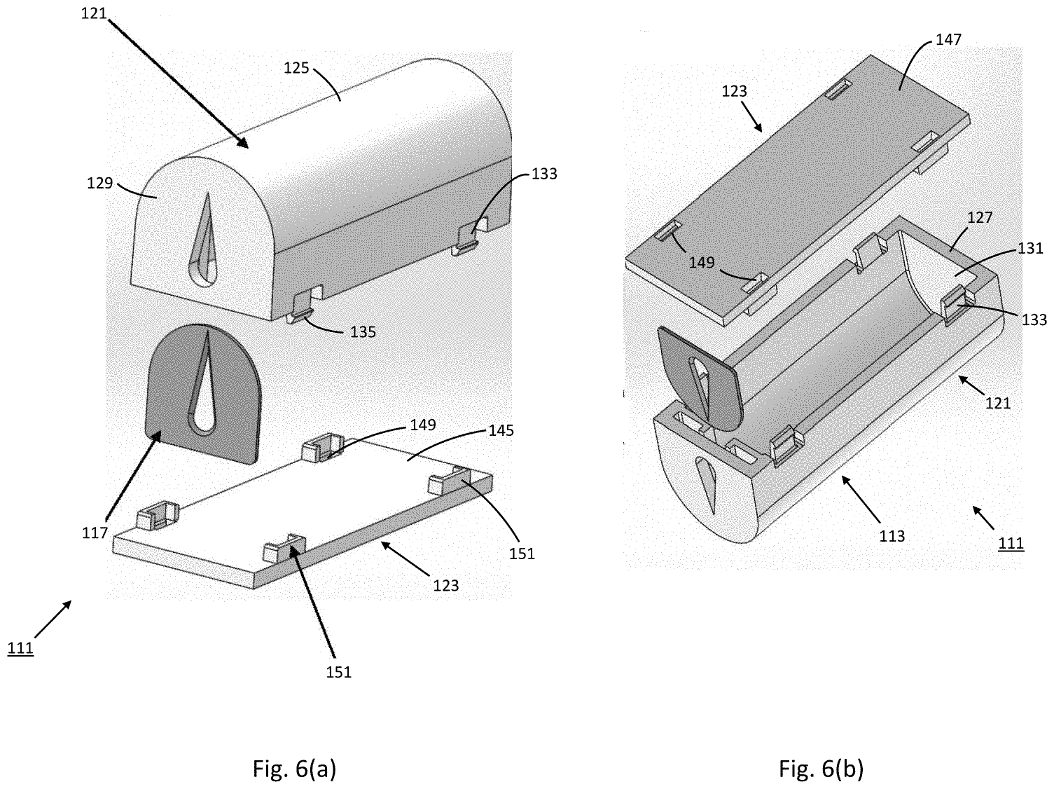

Referring now to FIGS. 6(a) and 6(b), outer housing 113 is similar to outer housing 13 in that outer housing 113 is a highly compact, pocket-sized, stick-shaped member with a two-piece construction comprised of a hollow, hemicylindrical cover 121 and a flat, plate-like base 123 which together define interior cavity 115.

Hemicylindrical cover 121 is similar in construction to cover 21 in that cover 121 is formed as a generally hollow member which comprises a rounded top surface 125, an open bottom surface 127, a front face 129, and a rear face 131. Cover 121 differs primarily from cover 21 in that a plurality of articulating locking pawls 133 is integrally formed in bottom surface 127 and project outwardly therefrom. Each locking pawl 133 includes an enlarged, ratchet-shaped head 135 at its distal end, the function of which is to become apparent below.

Base 123 is similar to base 23 in that base 123 is formed as a unitary, plate-like member which comprises a generally flat top surface 145 and a generally flat bottom surface 147. Base 123 differs from base 23 in that base 123 includes four vertical bores, or thru-holes, 149. A U-shaped alignment boss, or post, 151 protrudes upward from top surface 145 along the outer periphery of each bore 149.

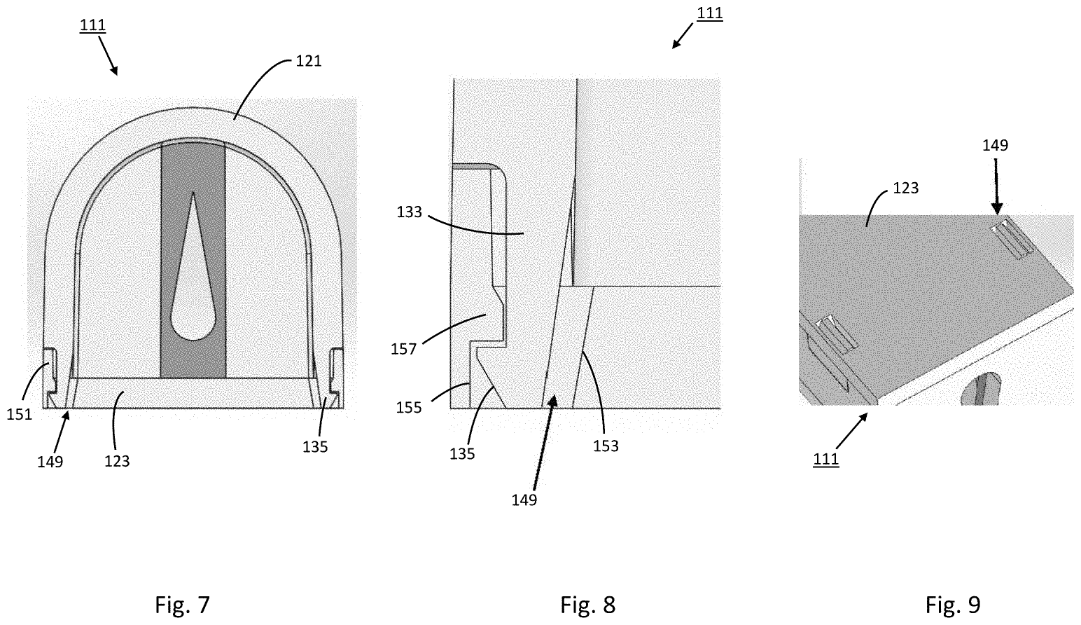

As seen most clearly in FIGS. 7 and 8, each thru-hole 149 is bordered by an inwardly sloped, or tapered, inner wall 153 and a generally vertical outer wall 155. Further, a rigid locking finger 157 is integrally formed in inner wall 155 and projects into thru-hole 149.

In this manner, each articulating locking pawl 133 is designed to penetrate down through a corresponding bore 149, with the clearance afforded by tapered inner wall 153 (approximately 0.015 inches) enabling enlarged head 135 on locking pawl 133 to extend beyond rigid locking finger 157. Once positioned beyond locking finger 157, the resilient construction of locking pawl 133 articulates locking pawl 133 outward, with enlarged head 135 positioned in direct contact against the underside of rigid locking finger 157. As such, cover 121 is snap-fit mounted onto base 123 and is incapable of removal therefrom. To further secure cover 121 onto base 123, an adhesive or similar bonding agent may be deposited into each thru-hole 149 from the underside of base 123, as can be envisioned in FIG. 9, thereby preventing any inward articulation of locking pawls 133 that may result in dissociation of housing 113.

It should be noted that, with cover 121 mounted onto base 123, each U-shaped alignment boss 151 fittingly projects into the void, or notch, formed in surface 125 of cover 121 due to the inclusion of each locking pawl 133, as seen most clearly in FIGS. 5 and 7. As a result, each boss 151 lies flush with top surface 125 of cover 121 to provide a uniform, finished exterior.

The invention described in detail above is intended to be merely exemplary and those skilled in the art shall be able to make numerous variations and modifications to it without departing from the spirit of the present invention. All such variations and modifications are intended to be within the scope of the present invention as defined in the appended claims.

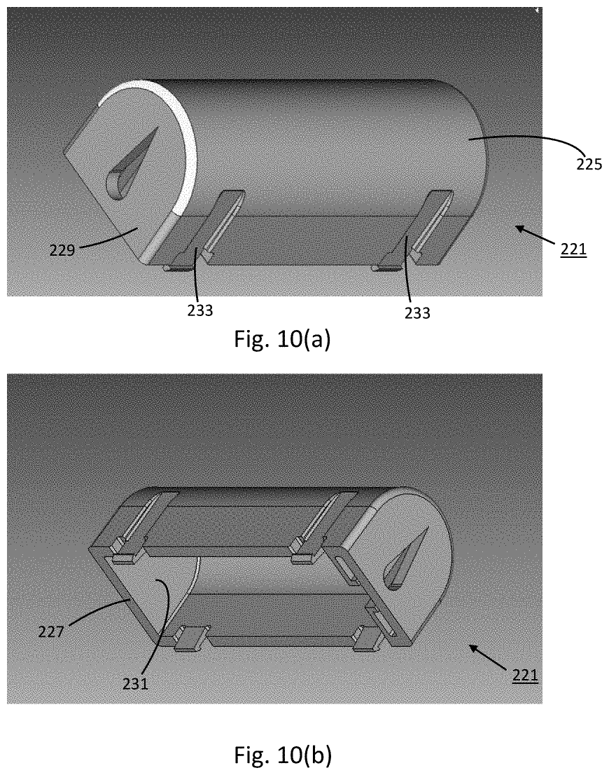

For instance, as a further design modification to the present invention, FIGS. 10(a) and 10(b) top and bottom perspective views, respectively, of an alternate embodiment of cover 121, the modified cover being identified generally by reference numeral 221. As can be seen, cover 221 is similar to cover 121 in that cover 221 is formed as a generally hollow member which comprises a rounded top surface 225, an open bottom surface 227, a front face 229, and a rear face 231. Cover 221 is also similar to cover 121 in that cover 221 includes a plurality of articulating locking pawls 233 which are integrally formed in bottom surface 227 and project outwardly therefrom.

Cover 221 differs from cover 121 in that each articulating locking pawl 233 is considerably longer in length, extending the majority of the height of cover 221. As can be appreciated, the increased length in pawl 233 greatly facilitates manufacturing of cover 221.

### Operation of Needle Tip Receptacle 11

In use, needle tip receptacle 11 is designed to operate in the following manner. Referring back to FIG. 1, receptacle 11 is constructed as a highly compact and portable device. As a result, it is envisioned that receptacle 11 could be habitually carried on the person of an individual who utilizes hypodermic syringes on a regular basis (e.g., in a clothing pocket).

Upon use of a hypodermic syringe, the user accesses receptacle 11 and inserts the distal end of the used syringe needle through opening 41 in front face 29 of cover 21 and, in turn, through opening 61 in knife blade 17. Positioned as such, the needle tip of the used syringe is located within substantially enclosed interior cavity 15.

Thereafter, the user manipulates the used syringe such that the metal needle is drawn into direct contact against sharpened edge 63 of knife blade 17. By urging the metal needle against sharpened edge 63 with sufficient force, the needle tip is severed from the remainder of the hypodermic syringe. Accordingly, the needle tip, which poses the greater safety hazard, remains safely and securely collected within interior cavity 15. The remainder of the syringe, which does not pose a significant safety hazard, can be discarded through more traditional means.

The aforementioned needle severing process can be repeated, as necessary, using receptacle 11. It is envisioned that receptacle 11 could be used to collect several used needle tips. Once full, receptacle 11 and the used needle tips retained therein can be collectively discarded via traditional biomedical waste disposal services. Thereafter, the user can utilize a new receptacle 11 for any future syringe disposal purposes.

The invention described in detail above is intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A receptacle for severing and retaining the needle tip of a hypodermic syringe, the receptacle comprising:
(a) an outer housing shaped to define an interior cavity; and
(b) a knife blade mounted within the interior cavity of the outer housing;
(c) wherein the knife blade is adapted to fragment the hypodermic syringe into a severed needle tip and a tip-less syringe, the severed needle tip being retained securely within the interior cavity of the outer housing.

2. The receptacle as claimed in claim 1 wherein the outer housing is shaped to define an opening in communication with the interior cavity.

3. The receptacle as claimed in claim 2 wherein the opening in the outer housing is adapted to receive the needle tip of the hypodermic syringe.

4. The receptacle as claimed in claim 3 wherein the outer housing comprises:
(a) a base; and
(b) a cover mounted onto the base, the cover and the base together defining the interior cavity of the outer housing.

5. The receptacle as claimed in claim 4 wherein the cover and the base are permanently secured together.

6. The receptacle as claimed in claim 5 wherein the cover and the base are permanently secured together by at least one of the group consisting of ultrasonic welding, adhesive bonding, and snap-fit interconnection.

7. The receptacle as claimed in claim 5 wherein the base comprises at least one boss for facilitating proper alignment of the cover onto the base.

8. The receptacle as claimed in claim 2 wherein the housing is shaped to define a slot which is dimensioned to fittingly receive the knife blade.

9. The receptacle as claimed in claim 8 wherein the knife blade is shaped to define an opening which is adapted to receive the needle tip of the hypodermic syringe.

10. The receptacle as claimed in claim 9 wherein the knife blade comprises a sharpened edge which immediately defines the opening in the knife blade.

11. The receptacle as claimed in claim 10 wherein the opening in the knife blade and the opening in the housing have similar dimensions.

12. The receptacle as claimed in claim 11 wherein, with the receptacle in its assembled state, the opening in the knife blade directly aligns with the opening in the outer housing.

13. The receptacle as claimed in claim 12 wherein the knife blade comprises a flat metal plate which is shaped to define the opening in the knife blade.

14. A method for severing and retaining the needle tip of a hypodermic syringe, the method comprising the steps of:
(a) providing a receptacle comprising an outer housing shaped to define an interior cavity and a knife blade mounted within the interior cavity of the outer housing, each of the outer housing and the knife blade being shaped to define an opening in communication with the interior cavity, the opening in the knife blade being immediately defined by a sharpened edge;
(b) inserting the needle tip of the hypodermic syringe through the opening in the outer housing, through the opening in the knife blade, and into the interior cavity; and
(c) manipulating the hypodermic syringe into contact with the sharpened edge in the knife blade so as to fragment the hypodermic syringe into a severed needle tip and a tip-less syringe, with the severed needle tip remaining inside the interior cavity in the outer housing.

15. The method as claimed in claim 14 further comprising the step of removing the tip-less syringe from the receptacle.

16. The method as claimed in claim 15 wherein the outer housing comprises:
(c) a base; and
(d) a cover mounted onto the base, the cover and the base together defining the interior cavity of the outer housing.

17. The method as claimed in claim 16 wherein the cover and the base are permanently secured together.

18. The method as claimed in claim 17 wherein the cover and the base are permanently secured together by at least one of the group consisting of ultrasonic welding, adhesive bonding, and snap-fit interconnection.
